(19) United States
(12) Patent Application Publication
(10) Pub. No.: US 2005/0222610 A1
(43) Pub. Date: Oct. 6, 2005

(54) Method and apparatus for performing septal surgeries

(76) Inventor: Jeremy S. Melker, Gainesville, FL (US)

Correspondence Address: BEUSSE BROWNLEE WOLTER MORA & MAIRE, P. A., 390 NORTH ORANGE AVENUE, SUITE 2500, ORLANDO, FL, 32801, US

(21) Appl. No.: 11/081,408

(22) Filed: Mar. 16, 2005

Related U.S. Application Data
(60) Provisional application No. 60/553,501, filed on Mar. 16, 2004.

(51) Int. Cl. A61B 17/44

(52) U.S. Cl. 606/205

(57) ABSTRACT

Disclosed herein are instruments and methods useful in performing nasal surgeries. Some instrument embodiments disclosed herein include a first arm and second arm, wherein said first and second arms are configured and sized for entry into a patient's nostrils and wherein distal portions that comprise guide channels defined thereon such that when said distal portions are closed onto a target site they form a continuous guide channel for guiding a needle.

This application claims the benefit of U.S. Ser. No. 60/553,501 filed Mar. 16, 2004

### BACKGROUND OF THE INVENTION

The septum is the cartilaginous wall which partitions the interior of the nose. The septum can be damaged by various agencies. Perforation of the septum is not uncommon and may result from, for example, physical injury and trauma, diseases, (e.g. syphilitic or tubercular ulceration), tumors, abscesses, and inhalation of corrosive chemicals and fumes.

Septal surgery was first described in 1882, and the techniques have been modified up to the present day to correct the septum and preserve as much cartilage as possible to prevent loss of support and saddling of the external nose. Submucous resection and septoplasty are performed for nasal obstruction, sinusitis, and headache. It is often performed in conjunction with rhinoplasty for cosmetic results. Septal surgery is among the most common operations performed. It is estimated that 500,000 septal surgeries are performed each year in the United States by Otolaryngologists and Plastic Surgeons.

Traditional septal surgery begins with an intranasal incision through the lining of the septum. The lining is then elevated away from the supporting skeleton by creating a tunnel on one or both sides of the septum. This allows direct visualization of the distorted bone or cartilage. Repair consists of removing or contouring the bone and cartilage. Support is assured by preserving as much cartilage as possible or by replacing crooked pieces of cartilage with straight pieces. Current practice involves removal of the deviated portion of the septum after which two preserved and opposing mucosal flaps are reapproximated to hasten healing and prevent hematoma. This reapproximation is typically attempted by packing or suturing. However, intranasal packing is generally left in place 24-72 hours and causes a great deal of discomfort to the patient. Further, studies have shown that intranasal packing reduces oxygen saturation and can cause toxic shock syndrome. Therefore, intranasal packing is not a benign procedure. Unfortunately, its alternative, intranasal suturing, is technically difficult and sometimes impossible to perform. Typically, the intranasal suturing is performed with a running “quilting” suture procedure. In the tight confines of the nasal with limited visualization, this portion of the procedure can be difficult and time consuming. Further, the lateral nasal sidewalls may be injured by the tip of the needle during the quilting process, leading to raw surface areas which are prone to scarring to the septum during healing. Such scars, called synechia are the most common complication of the procedure and may limit postoperative improvement in nasal breathing.

Accordingly, there is a need in the art for new devices, system, and methods of performing septal surgeries that address the aforementioned problems.

### SUMMARY

The present invention pertains to a novel surgical instrument embodiments for performing intranasal surgeries, and new methods of using such instrument embodiments. According to one aspect, the subject invention pertains to an instrument comprising a first arm assembly and an opposing second arm assembly wherein said arm assemblies are interconnected at a base. The instrument comprises a plurality of fasteners that are disposed at one or both of said arms. The instrument comprises the appropriate mechanical components and actuating mechanism such that upon actuation, said first arm and second arm are brought together, with a portion of patient tissue in between (preferably two planar portions of tissue), and said fasteners are secured to such tissue. In a preferred aspect, the instrument secures together two mucosal flaps that are formed during a septal surgery. Preferred still, the instrument comprises a handle assembly that is coupled to the proximal portions of the first and second arm assemblies, such that manual actuation of the handle assembly moves the distal portions of the first and second arm assemblies towards each other. The instrument provides for a plurality of fastening points dispersed within the target tissue so as to provide increased structural support through one actuation of the instrument.

Thus, an object of the invention is to provide an intranasal fastener driving device which can be accommodated by the narrow nasal passages to perform a fastening process and then be removed easily from the passages. Fastener(s) may be sutures, staples, rivets or similar surgical fasteners, preferably those that are bioabsorbable. According to a specific embodiment, the surgical instrument comprises a housing that contains a plurality of fasteners which is positioned on one or both of the arm assemblies, e.g., a conventional surgical stapler apparatus having a cartridge housing, or similar means, is described in U.S. Pat. Nos. 6,131,789, 4,241,861; or 5,655,698 and which is modified such that the cartridge housing comprises the proper dimensions and shape for intranasal insertion. Naturally, there would be no need for a linear cutting feature that is typically provided with commercially available surgical staplers. When the device is properly positioned alongside the target region of the nasal septum, the device is actuated or fired such that the two arms are brought together and the cartridge ejects the fasteners which are secured to the target tissue.

In a separate embodiment, the invention pertains to a surgical instrument that comprises a two-part guide frame that comprises a first arm and an opposing second arm wherein said arms are interconnected at a base. The instrument comprises a handle that is coupled to the proximal portions of the first and second arms, such that manual actuation of the handle assembly moves the distal portions of the first and second arms towards each other. When the first and second arms are brought together, they form a surgical guide for facilitating suturing of the tissue disposed between the arm distal portions. Using a flexible needle (or similar device) attached to a suture, the guide frame allows a surgeon performing septoplasty to expediently perform a stitch pattern (e.g. “quilt pattern”) for securing together two mucosal flaps.

According to other embodiments, the invention pertains to methods of utilizing the novel surgical instruments herein in performing nasal surgeries. Further advantageous aspects of the subject invention are described below.

### BRIEF DESCRIPTION OF THE DRAWINGS

FIG. 1 represents in diagram form a common septal surgery for correcting a deviated septum. FIG. 1(a) shows a side view exposed portion of a patient's target surgical site. FIG. 1(b) shows a cross-sectional view of the deformed septal region. FIG. 1(c) shows a cross-sectional view of the surgical area with the deformed cartilage removed, thereby leaving two portions of mucosa with a space between them. FIG. 1(d) shows a cross-sectional view of the typical suturing pattern to secure together the two mucosal flaps. FIG. 1(e) shows a side view of the secured tissue.

FIG. 2 shows an embodiment of the subject invention and an example, in diagram form, of its operation during a septal surgery. FIG. 2(f) shows a perspective side view of such embodiment. FIG. 2(a) shows the embodiment inserted into the nasal passages of a patient. FIG. 2(b) shows a cross-sectional view of the instrument embodiment positioned over the target tissue area. FIG. 2(c) shows a cross-sectional view of the closing of the embodiment to secure together two portions of a patient's tissue. Figure (d) shows a side view of the secured tissue, which illustrates the pattern of fasteners inserted into the tissue.

FIG. 3 shows a guide instrument embodiment of the subject invention and method of use. FIG. 3(a) shows a side view of a flexible needle secured to a suture for use with this embodiment. FIG. 3(b) shows a partial cross-sectional view of the embodiment closed around a target tissue site. FIG. 3(c) shows a cross-sectional view of the flexible needle being inserted through the guide embodiment.

FIG. 4 represents a side planar view of the embodiment shown in FIG. 3. FIG. 4(b) shows a side view of the inside surface of the distal portion of one arm of the embodiment. FIG. 4(c) shows a side view of the inside surface of the distal portion of the opposing arm. FIG. 4(a) shows a side view of the suture pattern produced by the embodiment.

FIG. 5 a side view of another embodiment of the subject invention.

FIG. 6 shows an embodiment of the subject invention comprising a mechanism to urge the needle, or similar means through the tunnels.

FIG. 7 shows an embodiment of the subject invention comprising an alternate mechanism to urge the needle, or similar means through the tunnels.

### DETAILED DESCRIPTION

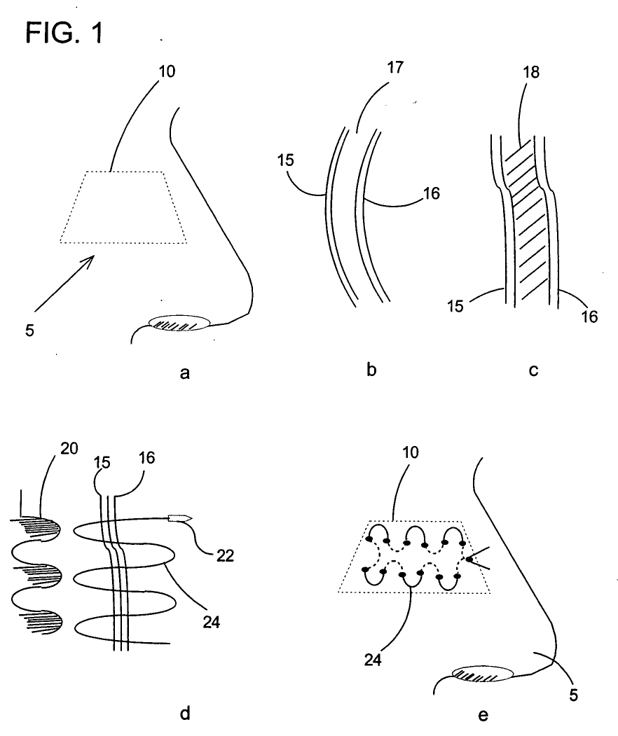

Turning now to the drawings, FIG. 1 illustrates the conventional quilt stitch method typically used in septoplasty. FIG. 1(a) shows the surgical site of 10 of a patient's septum 5. FIG. 1(b) shows the deviated septum 17 with opposing mucosa layers 15, 16. Upon correcting or removing the defected septum 17, a space 18 is created between the opposing mucosa layers 15, 16. See FIG. 1(c). It is important to close this space 18 to prevent surgical complications such as formation of a hematoma. FIG. 1(d) shows the typically stitching pattern wherein a needle 22 conjoined to a suture 24 is passed back and forth through the opposing mucosa layers 15, 16. The mucosa layers 15, 16 are brought together by the stitching thereby eliminating the undesired space created by the earlier part of the surgery. As discussed above, the tight confines of the nasal passage make this stitching procedure difficult. Further, there is a likelihood that the needle can scrape or puncture the lateral nasal sidewall. This can lead to raw surface areas which are prone to scarring to the septum during healing. Such scars, called synechiae are the most common complication of the procedure and may limit postoperative improvement in nasal breathing.

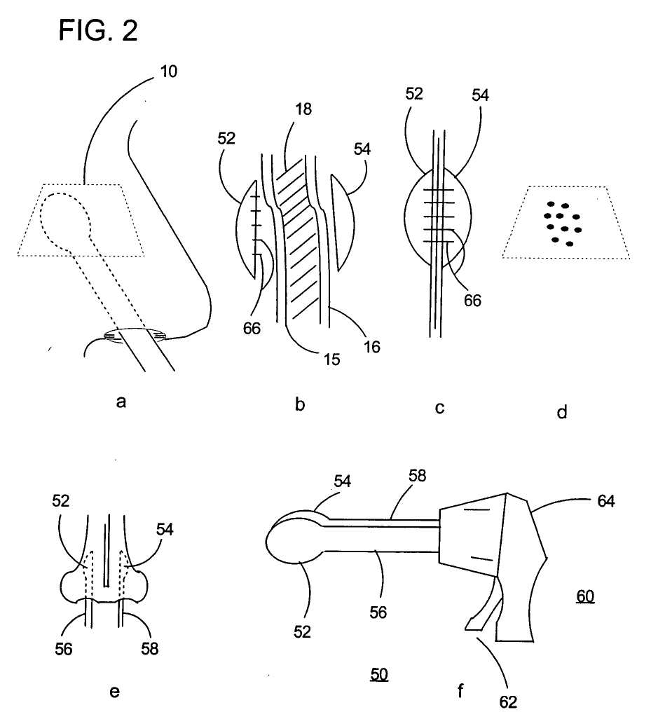

FIG. 2 illustrates a basic embodiment according to the principles of the subject invention. A surgical instrument 60 is shown comprising a first arm 56 and a second arm 58 which are interconnected by a handle assembly 64. The arms 56, 58 are inserted into the nasal passages of the patient and positioned over the target area. The handle assembly comprises a handle actuator 62 which when squeezed brings the first arm 56 and second arm 58 together. The first and second arms 56, 58 comprise distal portions 52, 54, respectively. Distal portion 52 has disposed therein a plurality of fasteners 66. When the distal portions 52, 54 are brought together, the fasteners 66 are ejected such that they penetrate the mucosal flaps 15, 16. The fasteners are aligned so that they create a generally oval shaped pattern in the mucosal flaps 15, 16.

The fasteners are preferably bioabsorbable staples, but those skilled in the art will appreciate that the fasteners implemented may be any suitable bioabsorbable, polymer based, or metallic staples, sutures, or the similar fasteners conventionally used in surgical procedures. There are numerous surgical stapling devices that are provided in the prior art. The inventor is not limited to one surgical stapling mechanism for fastening together the opposing mucosa layers 52, 54.

It is to be understood that numerous stapling mechanisms may be adapted for use in accord with the instruments and methods described herein. Examples of stapling instruments and stapling mechanisms that may adapted include, but in no way are limited to, those described in U.S. Pat. Nos. 4,241,861; 5,655,698; 4,892,244 and the references cited therein (all incorporated by reference), among many others. The critical feature of the stapling device to achieve and carry out the methods disclosed herein is that it must possess the proper size and dimensions so as to be inserted into the nasal passages of the patient to the targeted surgical site. Preferably the arms are slightly curved, or the dimensions are modified, so as to not damage the septum or other tissue between the arms that is not being fastened together, but which is nonetheless positioned between the arms of the instrument. In other words, fastening may occur at the distal portions of the arms without damaging tissue positioned between the intermediate portions of the arms. Preferably still, the fasteners are aligned in the instrument to mimic the quilt-type suture pattern typically used in septoplasty, i.e., a generally rectangle or oval pattern.

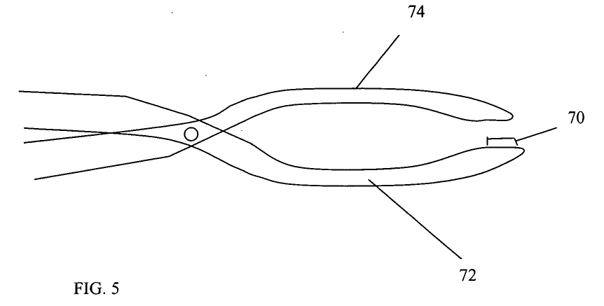

For example, an embodiment similar to that disclosed in U.S. Pat. Nos. 5,655,698 and 4,241,861 is modified such that a staple cartridge loaded with a plurality of bioabsorbable staples has the appropriate size, dimensions and configuration to pass through the intranasal passages as shown in FIG. 5. The distal portion 73 of arm 72 has staple cartridge 74 positioned thereon. The opposing arm 75 comprises a distal portion 76, which has disposed thereon grooves positioned and aligned with the staple cartridge so that each groove acts as an anvil for the correspondingly aligned fastener (not shown). Given the diameter of the mucosal flaps to be secured, in most cases, the cartridge does not need to possess a firing means (such as springs or the like) to eject the fasteners through the tissue. In most cases, sufficient force may be applied manually to eject the ends of the fasteners, causing them to curve around in the anvil grooves of the opposing distal portion 76. However, clearly such firing mechanism may be implemented in the cartridge. Further, the arms 72 and 75 are curved so as to avoid damaging the intervening tissue upon applying pressure to the distal portions 73 and 76.

Those skilled in the art, in view of the teachings herein, will note that the fasteners may take several different forms, and that the form will depend on the characteristics of the fastening material used. The fasteners may take the form of conventional surgical staples. Alternatively, the fasteners may take the form of rivets. In the case of rivets, one distal portion comprises the male rivet portion and the opposing distal portion comprises the receiving female rivet portion that is securable to the male rivet portion.

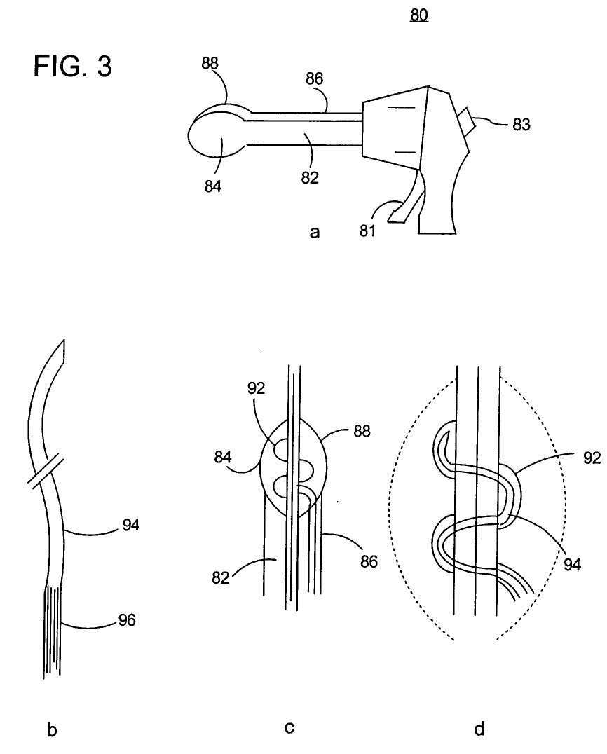

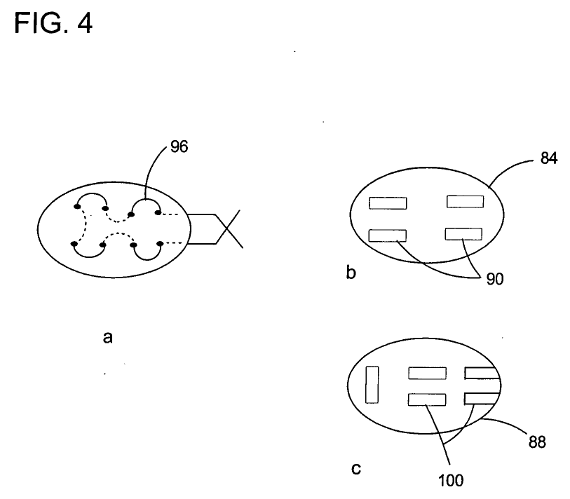

Next, turning to FIGS. 3 and 4, a surgical guide embodiment 80 is shown which comprises a first arm 82 having a distal portion 84 and a second arm 86 comprising a distal portion 88. The distal portions 84, 88 shown in FIG. 3 have a paddle-like shape, but may take any number of suitable configurations. The distal portions 84, 88 comprise guide channels 90, 100 defined thereon. The guide channels 90, 100 are aligned such that when the distal portions 84 and 88 are brought together, they define a continuous channel 92 for directing a flexible needle 94 (e.g. guidewire) attached to a suture 96. The flexible needle is inserted into a port (not shown) defined on the outside surface of the first distal portion 84 and the needle is pushed by the surgeon through the channel defined by the guide channels 90 defined on the opposing distal portions 84, 88. The channels 90, 100 can be semi-arcuate grooves which are defined on the inside surfaces of the distal portions 84 and 88.

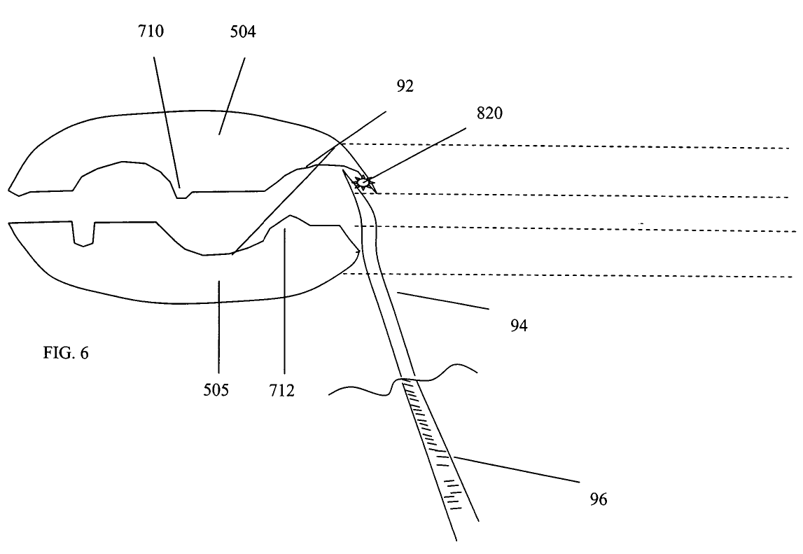

In a preferred embodiment, cross section as shown in FIG. 6, the distal portion 504 comprises a means for urging the flexible needle 96 through the channel 92. The embodiment shown comprises a rotatingly engaging feature 820 ratchet mechanism disposed at its first tunnel 126. Preferably the rotatingly engaging feature 820 is made of rubber, silicon, plastic or similar material such that it urges the flexible needle 96 by a frictional contact. The rotatingly engaging feature may comprise a plurality of extensions to increase the frictional contact with the flexible needle 96. The rotatingly engaging feature 820 is in communication with other mechanical components interlinked (not shown) in the distal portion 504 and/or the corresponding arm such that when the actuator 81 is squeezed the rotatingly engaging feature 820 rotates, causing the flexible needle to move through channel 92. Those skilled in the art will appreciate that the rotatingly engaging feature may be configured in distal portion 505 and/or corresponding arm, or both distal portions 504, 505 and/or corresponding arms. In a preferred embodiment, the inside surface of distal portions 504 and 505 comprises protrusions, 710 and 712, respectfully, which act to influence the needle to enter the appropriate corresponding tunnel hole.

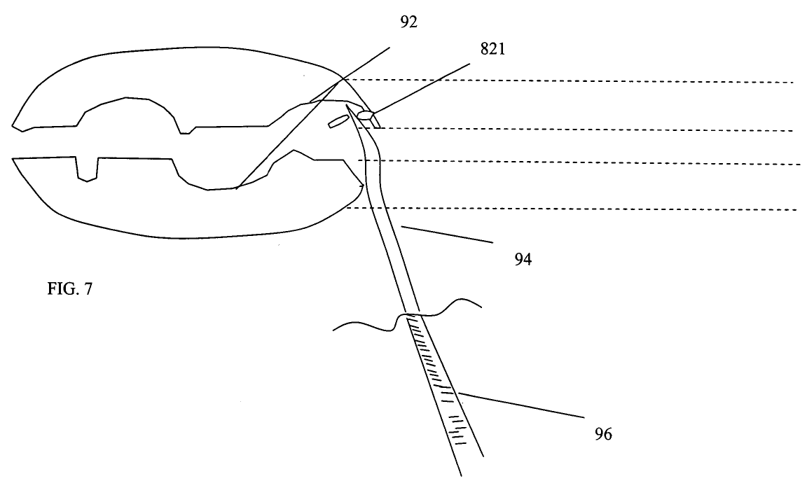

It is noted that the guide embodiment may comprise any of a number of different forms for the urging feature to urge the flexible needle through the channel. For example, the urging feature may take the form of two pivoting members 821 that grab the flexible needle 94, pivot, and release the needle. See cross-sectional view shown in FIG. 7. The pivoting mechanism occurs upon squeezing the actuator 81. After urging the needle forward through the channel 92 and releasing the flexible needle 94, the pivoting members 821 flip back to their starting position to repeat the urging effect upon subsequent actuation.

Furthermore, those skilled in the art will appreciate that the guide embodiment preferably comprises an actuation feature and a separate a closing feature. The closing feature will close the arms of the guide embodiment and secure the guide embodiment at the target site. A separate actuation feature will urge the flexible needle. The actuation feature and/or closing feature may be designed to be squeezed, pulled, depressed, flipped, turned and the like. For example, FIG. 3a shows a rotating knob 83 positioned on the handle assembly. The rotating knob 83 closes the arms 82, 86 when rotated. The actuator 81 urges the needle as described above. Alternatively, the actuator shown in FIG. 3a may be designed to close the arms 82, 86 and the rotating knob 83 may be designed to urge the needle through the embodiment.

Those skilled in the art will recognize that any number of mechanisms may be employed to urge the needle through the channel 92 such as, but not limited to, a roller communicatingly attached to a rotating knob, screw, tab etc., wherein manual rotation of such feature causes the roller to rotate thereby causing the needle to move through the channel 92.

According to another embodiment, the invention is directed to a method of performing septoplasty comprising obtaining an instrument comprising a first arm and second arm, wherein said first and second arms are configured and sized for entry into a patient's nostrils; positioning the instrument such that a distal portion of the first arm and a distal portion of the second arm are placed about a target site; actuating the instrument such that the first and second arms close onto the target site; and securing a plurality of fasteners into the target site in one motion. The step of positioning the instrument preferably occurs subsequent to some removal of tissue, or correction of tissue, at the patients nasal septum. The targeted area is preferably the nasal septum.

In a different embodiment, the invention is directed to a method of performing nasal surgery that comprises obtaining a surgical instrument comprising a first arm and second arm, wherein said first and second arms are configured and sized for entry into a patient's nostrils; positioning the instrument such that a distal portion of the first arm and a distal portion of the second arm are placed about a target site wherein the distal portions comprise guide channels defined thereon such that when said distal portions are closed onto a target site they form a continuous guide channel for guiding a needle; and urging a flexible needle through said continuous guide channel.

The teachings of the references cited throughout the specification are incorporated herein in their entirety by this reference to the extent they are not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims.

1. A surgical instrument that comprises a two-part guide frame that comprises
a first arm and an opposing second arm wherein said arms are interconnected at a base;
a handle that is coupled to the proximal portions of the first and second arms;
a closing feature that moves the distal portions of the first and second arms towards each other, wherein the distal portions comprise guide channels defined thereon such that when said distal portions are closed onto a target site they form a continuous guide channel for guiding a needle.

2. The surgical instrument of claim 1 further comprising an actuation feature that urges a flexible needle through the continuous channel upon actuation.

3. A surgical instrument that comprises a two-part guide frame that comprises
a first arm and an opposing second arm wherein said arms are interconnected at a base;
a handle that is coupled to the proximal portions of the first and second arms;
a closing feature that moves the distal portions of the first and second arms towards each other, wherein the distal portions comprise tunnels defined therein such that when said distal portions are closed onto a target site they form a substantially enclosed channel for guiding a needle; and
an actuation feature that urges a flexible needle through the continuous channel upon actuation.

4. A surgical instrument comprising
a first arm assembly and an opposing second arm assembly wherein said arm assemblies are interconnected and configured to fit into a patient's nostrils;
a plurality of fasteners disposed at a distal portion of one or both of said arms;
wherein upon said first arm and second arm being brought together over a target tissue site said fasteners are secured to said target tissue site.

5. A method of performing septoplasty comprising
obtaining an instrument comprising a first arm and second arm, wherein said first and second arms are configured and sized for entry into a patient's nostrils;
positioning the instrument such that a distal portion of the first arm and a distal portion of the second arm are placed about a target site;
actuating the instrument such that the first and second arms close onto the target site; and
securing a plurality of fasteners into the target site in one motion.

6. The method of claim 5, wherein said target site is a nasal septum.

7. A method of performing nasal surgery that comprises
obtaining a surgical instrument comprising a first arm and second arm, wherein said first and second arms are configured and sized for entry into a patient's nostrils;
positioning the instrument such that a distal portion of the first arm and a distal portion of the second arm are placed about a target site wherein the distal portions comprise guide channels defined thereon such that when said distal portions are closed onto a target site they form a continuous guide channel for guiding a needle; and
urging a flexible needle through said continuous guide channel.

8. The method of claim 7, wherein said target site is a nasal septum.
